(19) United States
(12) Patent Application Publication
(10) Pub. No.: US 2021/0007621 A1
(43) Pub. Date: Jan. 14, 2021

(54) METHOD TO ANALYZE CARDIAC RHYTHMS USING BEAT-TO-BEAT DISPLAY PLOTS

(71) Applicant: DP Holding (U.K) Limited, Grantham (GB)

(72) Inventors: Darren SPENCER, Oxfordshire (GB); Peter BALMFORTH, Oslo (NO)

(21) Appl. No.: 17/041,752

(22) PCT Filed: Mar. 27, 2019
(86) PCT No.: PCT/EP2019/057810

§ 371 Date: Sep. 25, 2020

(30) Foreign Application Priority Data
Mar. 27, 2018 (GB) 1804933.8

(51) Int. Cl. A61B 5/0468; A61B 5/0456; A61B 5/0452; A61B 5/044; A61B 5/0205; A61B 5/0408; A61B 5/0428; A61B 5/042; A61B 5/0404; G16H 50/30; G16H 50/20

(52) U.S. Cl. CPC A61B 5/0468; A61B 5/0456; A61B 5/04525; A61B 5/044; A61B 5/02055; A61B 5/021; A61B 5/04286; A61B 5/042; A61B 5/0404; G16H 50/30; G16H 50/20; A61B 5/04087

(57) ABSTRACT

Provided are controllers, methods and systems for monitoring cardiac data for the presence of arrhythmia. A controller as provided comprises input means arranged to receive first cardiac data corresponding to a time period, processing means and output means. The processing means is arranged to identify, within the first cardiac data, a plurality of events corresponding to ventricular contraction, and a time associated with each event; determine a plurality of intervals between the times associated with chronologically successive events; and produce second cardiac data in dependence on the determined intervals. The output means is arranged to transmit an output signal to a display means based on the second cardiac data, for displaying a beat-to-beat display plot corresponding to at least a part of the time period on the display means. A system as provided may comprise such a controller and a display means for displaying the beat-to-beat display plot corresponding to at least a part of the time period. Also provided are methods of analysing and monitoring cardiac data for the presence of arrhythmia, and non-transitory, computer-readable storage media which store instructions thereon that when executed by one or more processors causes the one or more processors to carry out such a method.

### FIELD

The present invention relates to a controller for the analysis of cardiac data such as electrocardiogram or pulse data.

### BACKGROUND

Many methods exist for gathering data associated with the function of the heart. In particular, it is possible to detect a subject's pulse in several ways, or to measure electrical activity associated with the function of the heart muscle itself, that is, electrocardiogram (ECG) data. Such information may be used clinically to detect, diagnose and monitor signs of healthy, atypical and/or pathological heart function. As technology improves, and particularly with the advent of personal devices which can continually monitor heart activity, large amounts of data have become readily available. However, determining the significance of such large amounts of data in an efficient way poses a challenge.

U.S. Pat. No. 9,408,545 B2 describes a computer-implemented method for efficiently encoding and compressing ECG data optimized for use in an ambulatory ECG monitor.

WO 2016126931 A1 describes a method for encoding and compressing ECG data comprising a first lossy process and a further lossless process.

WO 2016145392 A1 and U.S. Pat. No. 9,408,551 B2 are concerned with presenting R-R interval data in a format that includes relevant near field (short duration) and far field (medium duration) ECG data.

U.S. Pat. No. 9,504,423 B1 discusses a health monitoring apparatus and computer system adapted to download and evaluate ECG and physiological data against diagnostic criteria.

U.S. Pat. No. 9,619,660 B1 is concerned with a computer implemented system for secure physiological data collection and processing.

The present invention aims to provide means to convert recorded and/or real time electrocardiogram data, or pulse data, into timing intervals. Specifically the invention aims to use medium- and long-term ECG data or pulse data efficiently to detect, monitor and/or diagnose heart conditions.

### SUMMARY

In one aspect of the invention, there is provided a controller for monitoring cardiac data for the presence of arrhythmia, the controller comprising input means arranged to receive first cardiac data corresponding to a time period, processing means and output means. The processing means is arranged to identify, within the first cardiac data, a plurality of events corresponding to ventricular contraction, and a time associated with each event; determine a plurality of intervals between the times associated with chronologically successive events; and produce second cardiac data in dependence on the determined intervals. The output means is arranged to transmit an output signal to a display means based on the second cardiac data, for displaying a beat-to-beat display plot corresponding to at least a part of the time period on the display means.

The controller may further comprise a memory. The input means of the controller may be arranged to receive a request to alter the part of the time period to which the beat-to-beat display plot corresponds. Such a request may be for example in the form of one or more system generated command signals to alter the time period, or may encompass one or more user generated command signals received in response to a user entering a command signal via a user interface.

The first cardiac data may in some embodiments comprise ECG data. In this case, the processing means may be arranged to identify a plurality of R-waves in the ECG data as events associated with ventricular contraction. The input means may be arranged to receive a request to display the ECG data associated with a particular part of the time period. Alternatively, or additionally, the first cardiac data may comprise pulse data.

The input means may be arranged to receive supplementary cardiac data corresponding to the same time period as the first cardiac data, the first and supplementary cardiac data corresponding to different sources of cardiac data. In such cases, the processing means may be arranged to identify, within the supplementary cardiac data, a plurality of supplementary events corresponding to ventricular contraction, and a time associated with each supplementary event, and to compare the identified events with the supplementary events to determine if a given event correlates with a corresponding supplementary event, and where a correlation exists for a given event, to use the identified event to determine the plurality of intervals between the times associated with chronologically successive events. The processing means may be configured to reject events corresponding to ventricular contraction where a correlation does not exist for a given identified event and a corresponding supplementary event. The first cardiac data and supplementary cardiac data may correspond to pulse data and ECG data, in either order.

In some embodiments, the processing means is arranged to analyse at least the second cardiac data to detect the presence of one or more episodes of arrhythmia. The processing means may be arranged to undergo a learning process to refine this analysis of at least the second cardiac data. This may be accomplished by the input means being arranged to receive a further input comprising analysis of at least the second cardiac data and the processing means being arranged to use said analysis in the learning process.

The processing means may be arranged to identify a deviation of at least the second cardiac data from a standard profile, and a learning process comprising updating the standard profile may take place. The processing means may be arranged to identify a rate of change of the calculated heart rates over time above a threshold, and a learning process comprising updating the threshold may occur. The processing means may be arranged to categorise at least the second cardiac data into one or more rhythmic categories according to stored templates or statistical features corresponding to said rhythmic categories, and a learning process comprising updating the stored templates or statistical features may take place.

In an embodiment, the output means is further arranged to transmit an alert signal dependent on the detection of one or more episodes of arrhythmia.

In another aspect, the invention provides a method of analysing and monitoring cardiac data for the presence of arrhythmia. The method comprises obtaining first cardiac data; identifying within the first cardiac data a plurality of events corresponding to ventricular contraction and a time associated with each event; determining a plurality of intervals between the times associated with chronologically successive events; and producing second cardiac data in dependence on the determined intervals. The method also comprises outputting, based on the second cardiac data, an output signal based on the second cardiac data to a display means for displaying a beat-to-beat display plot corresponding to at least a part of the time period on the display means.

The method may further comprise a step of displaying, based on the output signal, a beat-to-beat display plot corresponding to at least a part of the time period. In an embodiment, the method comprises altering the part of the time period to which the beat-to-beat display plot corresponds, based on a received request.

The method may also comprise a step of displaying further data selected from galvanic skin response, body temperature, posture, blood pressure and/or vascular tone data, the further data corresponding to the time period of the beat-to-beat display plot.

The method may include a step of analysing at least the second cardiac data to detect the presence of one or more episodes of arrhythmia, and a learning process may occur to refine the analysis of at least the second cardiac data.

In yet another aspect, the above method may be facilitated by a non-transitory, computer-readable storage medium storing instructions thereon that when executed by one or more processors causes the one or more processors to carry out the method.

In another aspect of the invention, a system for monitoring cardiac data for the presence of arrhythmia is provided, comprising a controller according to any embodiment of the controller aspect described above, together with a display means for displaying the beat-to-beat display plot corresponding to at least a part of the time period.

The system may be arranged such that the input can receive a request to alter the part of the time period to which the displayed beat-to-beat plot corresponds. The system may further comprise at least one device for collecting first cardiac data. Where the first cardiac data comprises ECG data, and the device may be selected from: a Holter monitor, a cardiac patch, a 12-lead ECG, a 5-lead ECG, a 3-lead ECG; a loop recorder and an implantable loop recorder.

Where the first cardiac data comprises pulse data, the device may be selected from: a wrist-mounted device, an arm-mounted device, and a finger-mounted device. The device may collect said pulse data by one or more of: pulse oximetry, pulse photoplethysmography, blood pressure measurement and electrical measurement.

Where the first cardiac data comprises pulse data, this may comprise pulse wave data. A device measuring pulse wave data may be an ultrasound monitor. The processing means may be configured to calculate blood pressure measurements based on the pulse wave data.

It is contemplated that any of the above features may be used in combination with each other, except where otherwise specified.

### DRAWINGS

FIGS. 1 to 10 show illustrative examples of beat-to-beat display plots and associated ECG data characteristic of particular heart conditions.

FIG. 11 shows a flowchart demonstrating the process of recording, uploading, processing and accessing cardiac data according to an embodiment of the invention

FIG. 12 shows a flowchart demonstrating a process of filtering and analysing ECG data and determining interval data for plotting and display, according to an embodiment of the invention.

FIG. 13 shows an illustration of a normal ECG trace, with labels to indicate the characteristic parts of the trace.

FIG. 14 shows an illustration of a trace of pulse wave data.

FIG. 15 shows an illustration of an ECG trace, and of pulse wave data, wherein the two traces are correlated with each other.

FIG. 16 shows a schematic of a controller and a system according to an embodiment of the present invention.

### DETAILED DESCRIPTION

Heart rhythm disorders, also known as arrhythmias or rhythmic disturbances, are disruptions to the normal rhythmic working of the heart. Major types of arrhythmias include atrial fibrillation, atrial flutter, ventricular fibrillation, tachycardia and bradycardia conditions, and others.

The term ‘first cardiac data’ as used herein refers to data collected for use in the invention, and suitably comprises ECG data. It may be collected by any appropriate method such as ECG monitors, including portable or Holter monitors; and/or by devices for measuring pulse rate in a central or peripheral location, including so-called smart devices, which may use pressure detection, photoplethysmography and/or the detection of electrical activity. The first cardiac data comprises detectable events which correspond to ventricular contraction.

The term ‘second cardiac data’ as used herein refers to data produced by a processing means, in dependence on the determined intervals found from the first cardiac data. Typically, second cardiac data comprises heart rate data, which is calculated from the determined intervals.

Electrocardiography (ECG), the recording of the electrical activity of the heart, has been used for many years to monitor the heart, and is indispensable in the diagnosis of many heart conditions and defects. ECG recording measures the electrical signal generated by the propagation of ionic action potential currents in the heart fibres. Devices for measuring ECG data in a clinical setting include 12-lead, 5-lead, and 3-lead ECG devices. Portable devices for ECG, sometimes known as ‘Holter monitors’, are also known, and allow recording over a longer time period than stationary recordings. Devices for measuring ECG have been incorporated into adhesive patches for wearable, ‘on body’ and non-invasive recording of ECG; such devices are known and referred to herein as so-called ‘ECG patches’ or ‘cardiac patches’.

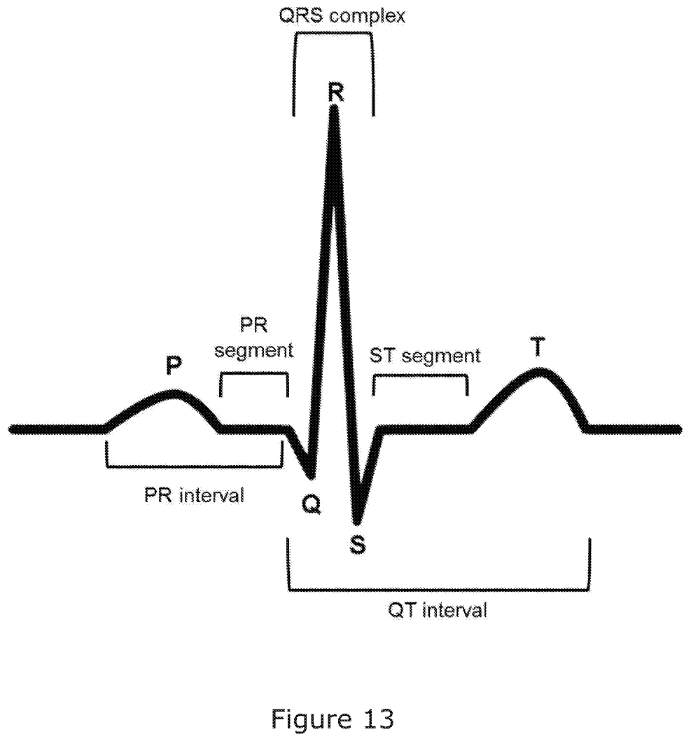

The classic features of an ECG trace are shown in FIG. 13. The trace includes characteristic ‘waves’, referred to by letters, which indicate particular electrical events taking place in the cardiac tissue. The P wave represents depolarisation of the atria, spreading from the sinoatrial (SA) node towards the atrioventricular (AV) node. This wave usually appears as a relatively slow positive wave. The QRS complex (sometimes referred to as the R-wave) represents depolarisation of the ventricles (which also corresponds to ventricular contraction), appearing as small negative deflections either side of a large positive signal. Finally, the slow positive T wave represents the repolarisation of the ventricles. The QRS complex has the largest amplitude due to the relative size of the ventricles, and due to this, the R-R interval, being the time between the R peaks of this complex, is often used to measure heart rate, which calculated by the inverse of the R-R interval. The interval between the P-waves of ECG traces is also sometimes used for the measurement of heart rate.

Disease states may change the appearance of these waves and the ECG is therefore heavily used in diagnosis. Some conditions require detailed investigation of individual features of the ECG, whereas some can be indicated by the R-R data alone, as discussed below.

Classic methods of inspecting ECG plots make it difficult to detect effects on a gross scale—it is not practical to look at more than a few tens of individual ECG traces at a time, and the classic ECG representation does not make effects on the R-R interval obvious. For example, ECGs are typically displayed at an effective paper speed of 25 millimetres (mm) per second, corresponding to 90 metres of recording per hour, when displayed in paper form. Even in electronic form, such an amount of data pose storage, transfer and recall problems, and are impractical to inspect. For these reasons, an ECG is not the best method for diagnosing arrhythmia conditions which show their effect over a long period of time. As discussed below, looking at accumulated data over a longer period—at a so-called ‘macro’ scale, more clearly shows changes in gross pattern, which can identify periods of interest for more detailed analysis on the ECG trace.

Cardiac Holter monitoring was established initially to help identify changes in the ST segment of an ECG trace following revascularization procedures (ischemia). Since these early days, cardiac Holter monitoring has advanced in its therapeutic uses, expanding to short duration arrhythmia monitoring (24 hours), but has been limited by device size and memory/recording capacity. Advances in miniaturisation, memory capacity, and patient wear times, has allowed for longer term data capture (greater than 24 hours). However, the quality of the data collected in this way can vary significantly; these recordings are prone to noise accumulation, from factors such as electrical or muscle stimulation, exercise, or equipment problems such as loose patches/electrodes, poor circuitry, and so on. This can mean that finer ECG details such as P waves are lost. The present invention is able to make use of events corresponding to ventricular contraction—that is, the R wave, which as the largest feature in an ECG trace are less easily corrupted. Current analysis software, for example the HE/LX® (helix) Analysis Software from North East Monitoring (NEMon), takes the raw data and typically analyses each cardiac beat and groups them together, looking at changes in morphology and rate to determine the presence of a cardiac arrhythmia or abnormality. With the increase in recording time, this dramatically increases the amount of time used to analyse each patient's individual recording, to determine the morphology and rate changes over the duration of the recording. From this, a report is created, with a gross determination of the general recording. The report highlights any potentially interesting changes in the electrocardiogram and provides short duration ‘snapshots’ (typically 8-30 seconds) to highlight the findings to the reader. As an example in a person with an average heartbeat of 60 beats per minute, this would display 5 to 30 cardiac cycles of information from an estimated 86,400 beats in one day, or just over 600,000 beats for a 7 day recording, thus presenting only a very small fraction of the available data at a time.

Typically Holter monitoring has been for 24-hour duration providing in the vicinity of 115,000 cardiac beats for analysis (based on an average heart rate of 80 beats per minute), or has been patient triggered, where the patient decides based on symptoms or otherwise when the data should be recorded for later analysis. While patient triggered data recordal cuts down on the total amount of recording for later analysis, the drawback is that many arrhythmic episodes are asymptomatic, and/or occur during sleep. The introduction of longer term and real-time monitoring exponentially increases the number of beats available for analysis, but also of course the time required to analyse the data.

The major challenge of current Holter monitoring analysis software is how to handle large data volumes for long term monitoring and to provide an accurate representation of the cardiac condition over the duration of the recording. In addition, even when large amounts of ECG data are available, analysis on a micro scale (8-10 beats at a time) is appropriate for detecting important, clinically diagnostic ECG morphology changes, but is less suitable for macro level information that, for example, may indicate not only an arrhythmia such as atrial tachycardia, but also allow rapid determination of factors such as the start (onset) and finish (offset) of the arrhythmic episode, as well as the maximum rate. These factors are of importance in making an accurate and useful diagnosis.

Other devices used in a clinical context include implantable loop recorders or insertable cardiac monitors, being devices which are inserted internally to continuously record a patient's heart activity. These recorders may store heart activity information for later review in response to a patient-delivered activation signal, for example the patient may trigger recording by pressing a button on a paired ‘activator’ device when they experience a fainting event, angina or other symptom, or the device may be programmed to record when it detects a heart rate above or below a certain rate. Any and all of the above-mentioned devices for collecting ECG data are contemplated for use in the current invention for the gathering of first cardiac data, along with any other suitable ECG-measuring device.

In addition to the above, devices exist for measuring heart rate, without use of ECG data, with so-called ‘smart watches’ and other products able to continuously monitor heart rate in real time using an array of methods, such as photo-sensitive sensors or detection of peripheral blood vessel pressure changes as well as standard and non-standard electrode-based systems of measurement. Such data which aims to measure heart rate without the use of ECG data is referred to herein as ‘pulse data’. The detection of a pulse by a suitable device of this kind indicates ventricular contraction. As a result, in many instances intervals between detected pulses correlate with intervals between R-waves in an ECG trace. As used herein, therefore, ‘R-R intervals’ and ‘R-R plots’ are also intended to encompass intervals and beat-to-beat display plots based on pulse data. Any suitable device for measuring pulse data is also contemplated for use with the invention, for example selected from wrist-mounted devices, arm-mounted devices, and finger-mounted devices. More generally, devices suitable for measuring pulse data via photoplethysmography can be positioned anywhere where a blood vessel can be measured, such as over the carotid.

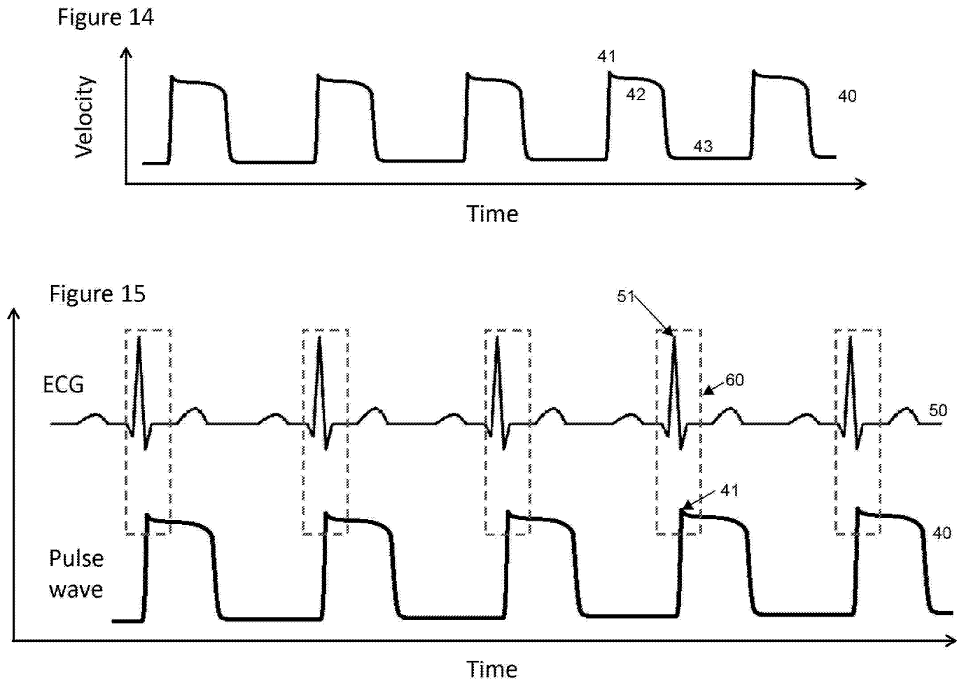

During contraction of the heart, a longitudinal pressure wave is created that propagates outwardly along the vessel walls of the vasculature. Based on this concept, in some embodiments, it is considered that pulse data can be gathered by measuring data relating to this pulse wave at one or more of a variety of body locations. Sensors can be placed and used to measure the arrival of the pulse wave. In particular, ultrasound methods of imaging blood vessels and blood flow can be used, for example by making use of the Doppler effect (Kisslo J A and Adams D B “Principles of Doppler Echocardiography and the Doppler Examination #1”. London: Ciba-Geigy. 1987). As shown for example in FIG. 14, a pulse wave form (40) at an arterial location can be measured overtime by monitoring the velocity of the underlying blood flow, and can display one or more peaks (41) on wave arrival, and a sustained level (42) during ventricular contraction, before dropping to a baseline level (43). By comparing the pulse wave arrival at different arterial locations (which gives a measure of pulse transit time, or PTT), or by correlating the pulse wave information with an accurate source of data measuring ventricular contraction (such as the R-wave of an ECG trace), it is possible to measure the velocity of the pressure wave, the so-called pulse wave velocity (PWV). This measure has been found to be directly proportional to blood pressure in many circumstances (Smith et al. Thorax 1999; 54:452-457), and so the monitoring of pulse wave information can also allow blood pressure to be calculated.

With the advent of improved sensing technology of any of the varieties discussed above, and better quality recordings of longer duration, the issue of how to precisely and accurately analyze and represent increased information load for greatest effect has not been addressed.

Current software (for example NEMon software) for analysing ECG data such as gathered by a Holter monitor typically analyses each beat of the recording and automatically compares beats against each other, then places each morphological type into a “bin” for further analysis. The cardiac beats in these bins are then analyzed based on duration or amplitude to determine if the beat is normal, supraventricular or ventricular in origin or is just noise, such as may be introduced by background, movement or muscular sources, or by the system itself. Each of these bins of individual beats will then needed to be verified as being correctly labeled, in order for the technician to make a diagnosis based upon a series of short duration segments (8-30 seconds) of cardiac beats.

Previous approaches (see for example WO 2016145392 A1 and U.S. Pat. No. 9408551 B2) have aimed to create reporting tools. Software has been used to focus analysis on micro-level data, that is, individual ECG traces of heartbeats, to attempt to identify changes at this level. After these analyses are carried out, reports are created which aim to give contextual macro-level data (such as R-R plots).

Comparing segments of cardiac rhythms of only short duration may typically provide information on the type of abnormal cardiac rhythm (the root of the problem); however by looking at only these short segments the overall picture of the cardiac function over time is lost. In conditions where rhythmic abnormalities are episodic, it may also be easy to overlook these episodes in longer recordings, especially if they are not associated with symptoms.

The goal of current Holter software systems is to provide a cardiac morphology analysis tool that helps the user automatically discriminate between normal and abnormal cardiac rhythms to create a report.

So-called Poincare plots are often used to illustrate the regularity of R-R intervals, and depict the duration of an R-R interval ‘n’ on the x-axis against the following R-R interval ‘n+1’ on the y-axis, thus indicating the degree to which R-R intervals are different from their immediate neighbours. This shows the variability of a heart rate, and can be useful in detecting the presence of rapid changes in heart rate and of particular rhythmic disturbances, such as atrial fibrillation.

In R-R plots, however, successive R-R intervals can be displayed on plots which thus show changes in heart rate over time. R-R plots (also referred to herein as beat-to-beat display plots) may also refer to plots where the inverse of the R-R interval is taken to calculate an instantaneous heart rate. Such plots can be useful in detecting times when the heart rate differs from normal over a longer time period than is immediately visible with a shorter time frame. The advantage of using an R-R plot over for example a Poincare plot is that the temporal relationship of a particular heart event is preserved, as well as the relationship with its immediate as well as more distant neighbours.

It is envisioned that R-R plots and other such beat-to-beat display plots may be delivered on a non-linear y-axis scale, to aid in visualisation. In particular, the plots may be delivered on a logarithmic scale on the y-axis (i.e., a semi-log scale), typically a log-2 or log-10 scale. The non-linear scale may be applied on part or all of the axis.

Additionally, the x-axis representing time may be non-linear. In particular, periods of time which are considered of less interest, for example periods which have been assessed by automatic analysis or the input of a medical practitioner to represent sinus rhythm may be compressed or excised, such that periods of more interest can be seen. In this way, different episodes of particular rhythmic disturbances can be directly compared to one another.

It is particularly envisioned that the time period to which the beat-to-beat display plot corresponds can vary. It should especially be possible for a user of the invention to refer to a particular display plot and then ‘zoom in’ on a particular time period to see it in more detail. In this way points of particular interest can be examined. In embodiments where the first cardiac data comprises ECG data, the invention may be configured to be able to display this ECG data when a request is made, or in response to a request for a time period to be displayed which is short enough that ECG data can be usefully analysed over such a period. Similarly, the user could ‘zoom out’ to see a longer time period, and thereby gain an overview of heart function over a greater period of time.

In this way, the display plots produced are intended to be interactive, such that the pertinent information may be selected with ease by a user of the invention, such as a medical professional, or a user of a fitness-monitoring system. In comparison to a system which would show a static snapshot of cardiac data, it is particularly advantageous to select, rapidly and straightforwardly, different time periods within what may be a recording of long duration, so that for example areas of interest can be rapidly identified in a display corresponding to a long time period, and those areas can be more closely studied in a display of a shorter time period. Additionally, due to the difference in utility in different contexts of larger scale ‘macro’ level data represented by the beat-to-beat display plot, and smaller scale ‘micro’ level data such as provided by ECG data, the ability to choose the nature of the displayed information allows efficient availability of the needed data at any time.

Certain arrhythmias may be relatively easily distinguished from a beat-to-beat plot due to their characteristic effects on the heart rate. In this way, parts of a beat-to-beat plot may be categorised as belonging to one or more ‘rhythmic categories’. Some non-limiting examples of these categories are described in greater detail below. Similarly, devices such as heart rate monitors (HRMs) that collect pulse data without concurrent collection of ECG data can be used as a diagnostic tool, due to macro level pattern recognition possible through the provision of beat to beat plots. Recently, it has been shown (see references 1, 2) that using data from pacemakers (measuring heart beat timing intervals rather than ECG data) can demonstrate that cycle length alone (that is, the duration from one measured variable to another such as the interval between ventricular depolarisations) can be used to diagnose and monitor an arrhythmia by applying standard mathematical indices of mean, mode, standard deviation, co-efficient of variance, and so on. These mathematical variables, combined with the visual macro level pattern recognition, are therefore able to be highly diagnostic and/or predictive of various types of arrhythmia. Such approaches are however dependent on the quality of the raw data. Thus by applying the correct filters at the front end, unnecessary elements can be removed (such as noise, wandering baselines, and so on) and so accurate timing intervals (P-P, R-R, P-R, P-T, R-T etc.) can be created, for optimal analysis of underlying rhythmic conditions.

Sinus rhythm is the normal functioning of a healthy heart, where the trigger for cardiac muscle contraction originates in the sinoatrial node and spreads through the heart. This leads to a regular contraction of the heart muscle. Sinus arrhythmia is where the interval between heartbeats varies, despite the trigger still occurring in the sinoatrial node. This can have a number of causes, such as respiration, or exercise. While mostly seen in younger healthy people, and generally of little concern, sinus arrhythmia can be a signal of heart disease, especially when it appears in the elderly.

Atrial fibrillation is a condition which causes an irregular and often abnormally fast heart rate, caused when the impulse generated by the sinoatrial node is overwhelmed by abnormal impulses generated elsewhere, such as in the roots of the pulmonary veins. It is the most common heart rhythm disturbance, affecting around one million people in the UK, and increasing in prevalence in older people. Atrial fibrillation is a marker for higher stroke risk. Episodes of atrial fibrillation may be marked by symptoms including heart palpitations, fainting, lightheadedness, shortness of breath, or chest pain, but in many cases episodes do not cause symptoms. Atrial fibrillation can be seen in an R-R plot as a ‘cloud-like’ dispersed pattern of irregular R-R intervals.

Atrial flutter is a condition which shows similar symptoms to atrial fibrillation and often occurs in the same patients. However, this condition is in some cases treated differently to atrial fibrillation, and as a result distinguishing between the two is important. Atrial flutter is characterised by rapid onset periods of an elevated heart rate, with a more regular rhythm than is usually found in atrial fibrillation. In an ECG trace, this condition can be determined from the appearance of characteristic ‘flutter waves’ or ‘F waves’, being a pattern of regular, rapid atrial waves at a regular rate of more than 200 per minute.

Bigeminy is a condition where there is a regular alternation of long and short heart beats, giving a regular pattern of grouped pairs of heart beats on an ECG trace. This condition is usually caused by ectopic heartbeats, that is, where the electrical trigger for cardiac muscle contraction originates outside the sinoatrial node. Trigeminy is a similar condition where triplets of heartbeats are seen.

Atrioventricular block (AV block) occurs when conduction between the atria and the ventricles is impeded, so decoupling the relationship between atrial and ventricular contraction. AV block takes several forms or degrees, for example, first degree AV block, which may be asymptomatic, is indicated by a P-R wave interval of more than 200 msec, while second and third degree blocks may interfere to a greater extent.

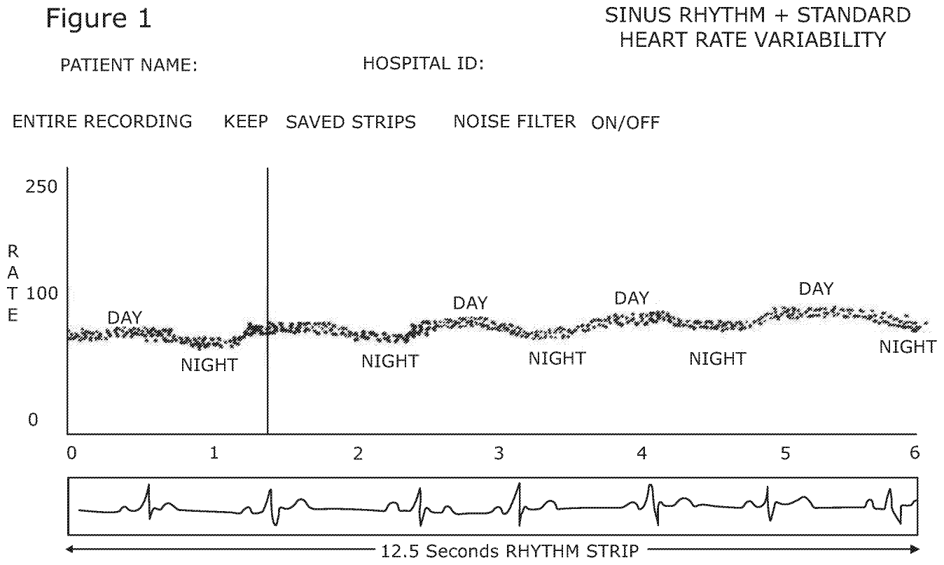

FIGS. 1-10 indicate illustrative examples of R-R plots (upper parts of each Figure) in accordance with embodiments of the invention demonstrating a number of types of heart activity, that is, different rhythmic categories, along with associated ECG information in a 12.5 second ‘rhythm strip’ in the lower panel. The vertical lines in the R-R plot and rhythm strip indicate the same time point and show from where the ECG data is taken.

FIG. 1 shows an illustration of sinus rhythm maintained over a period of several days, with higher heart rates during the day than the night. This sinus rhythm represents ‘normal’ cardiac activity, with an average heart rate of around 70 bpm, and a relatively low variability of this measurement.

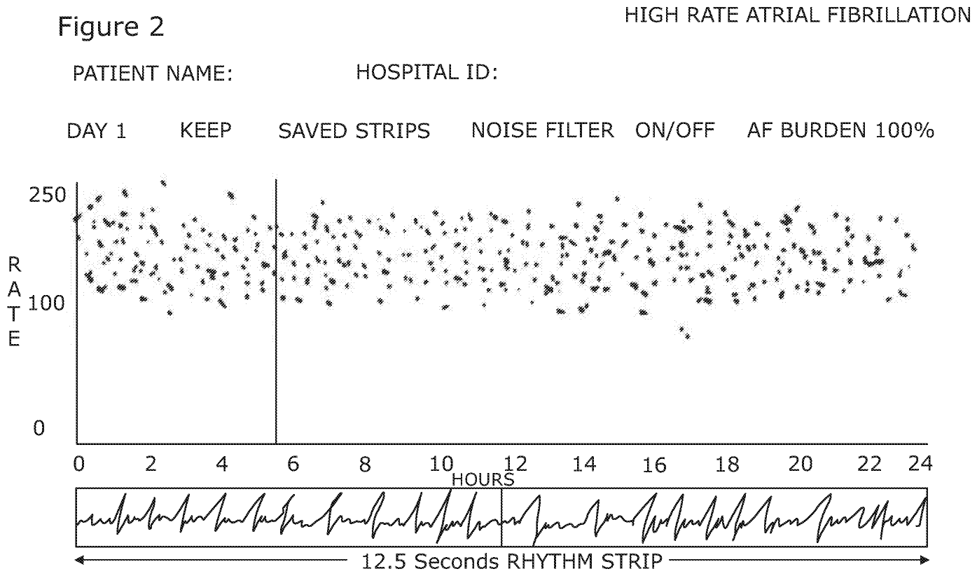

FIG. 2 shows an illustration of constant atrial fibrillation over a period of 24 hours (i.e. an atrial fibrillation burden of 100%). This is shown by the high and extremely variable heart rate over the entirety of this period. The illustrative ECG trace indicates a characteristic shape of atrial fibrillation.

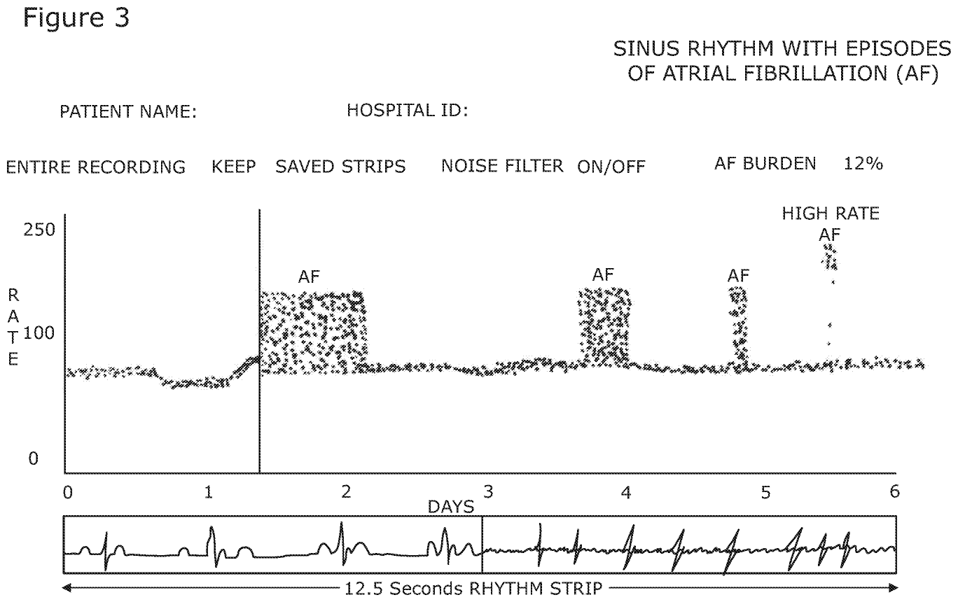

FIG. 3 shows an illustration of sinus rhythm with episodes of atrial fibrillation, or AF (an atrial fibrillation burden of 12%). The episodes of atrial fibrillation range between approximately 3 and 18 hours and are again indicated by a high and extremely variable heart rate. It is clear from the R-R plot that the onset of the atrial fibrillation episodes is rapid. This can be further confirmed by ‘zooming in’ on the onset point of the atrial fibrillation and obtaining the ECG trace associated with the beginning of the fibrillation episode, as shown in the rhythm strip of FIG. 3. This shows that the trace moves rapidly between a regular ECG trace on the left and the characteristic shape of a fibrillatory rhythm on the right.

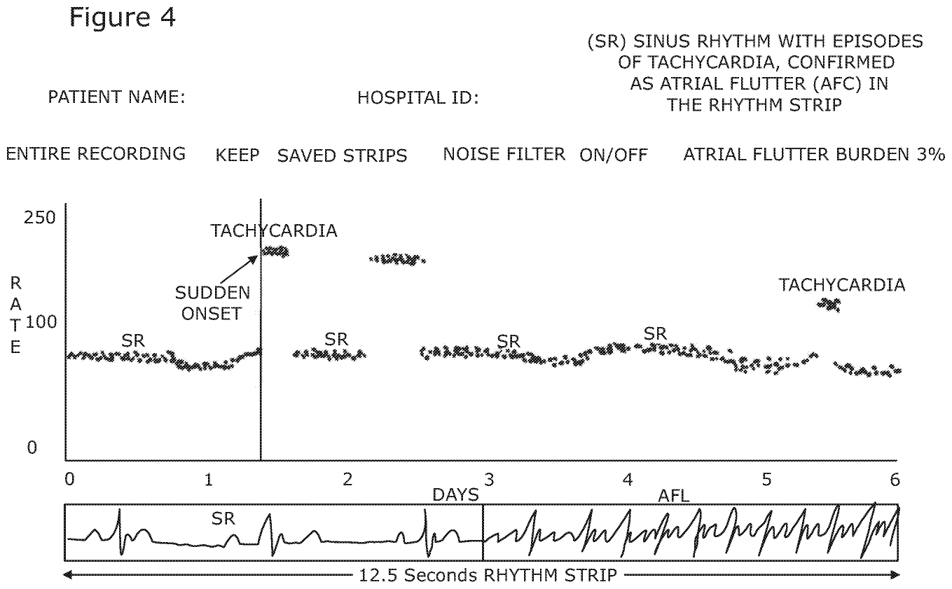

FIG. 4 shows an illustration of sinus rhythm with episodes of tachycardia. The R-R plot shows the baseline of sinus rhythm for the majority of the 6-day recording period, but with 3 episodes of high, but not especially variable, heart rate during this time.

The user of these data would be able to select the onset of the tachycardia episode, ‘zoom in’ on the R-R plot at this point and determine that the onset is rapid—there is little middle ground between the sinus rhythm and the episode of tachycardia. On zooming in further, the user would be able to access the associated ECG trace at the point of interest. This would further show the rapid onset of the episode, and would also show the characteristic features of atrial flutter, such as the appearance of ‘flutter waves’ or ‘F waves’, being a pattern of regular, rapid atrial waves, or a ‘saw tooth pattern’ of the P or F waves thus giving more detailed information on the particulars of the episode, and so aiding in diagnosis.

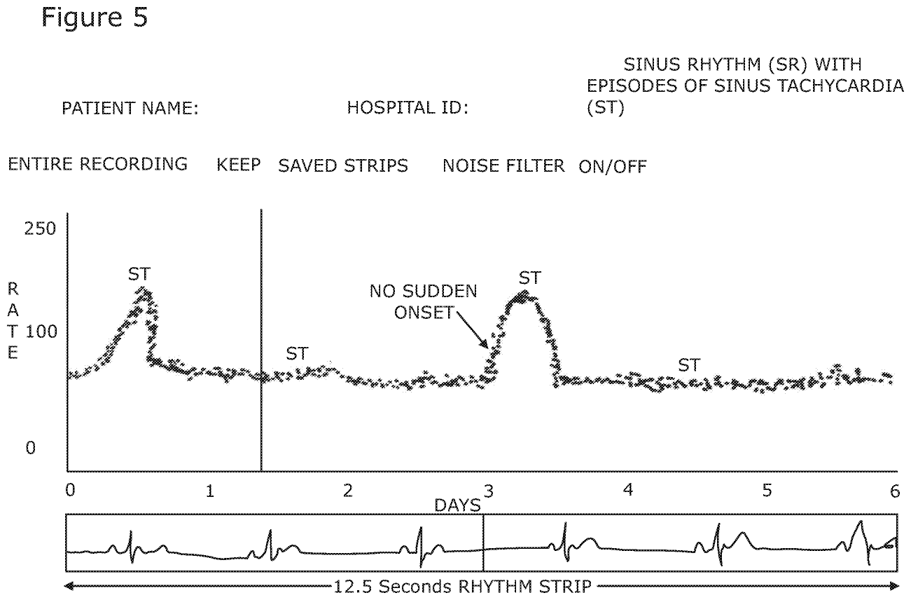

FIG. 5 shows another illustration of sinus rhythm with episodes of tachycardia over a six day period. It is clear from the R-R plot that these episodes are not rapid onset, but there is a smoother transition between sinus rhythm and tachycardia, thus indicating this is sinus tachycardia. While such episodes in healthy subjects are usually a result of normal physiological response to exercise or other stimuli, they may also indicate a pathological cause. As a result, this information may be used in combination with the ECG trace at this point, and/or with other data, such as a galvanic skin response or patient reporting, to determine if there is an underlying cause for the episodes.

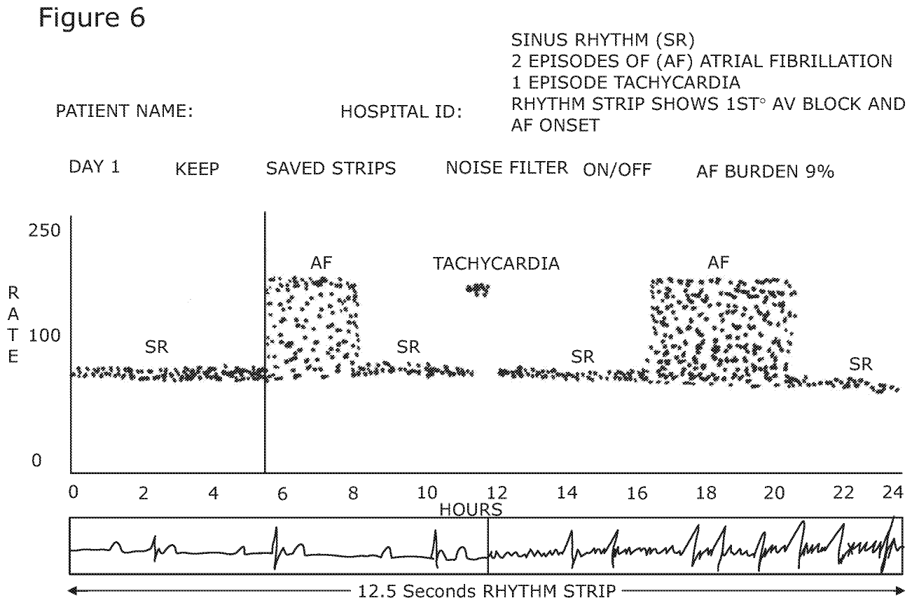

FIG. 6 shows an illustration of cardiac data over a 24 hour period with a mixed pattern of primarily sinus rhythm but with episodes of tachycardia and atrial fibrillation. The difference between the types of episode can be clearly seen with reference to the R-R plot, as well as the points at which the plot switches between them. The lower panel again illustrates the ECG trace which might be seen if the user of the data were to select the onset of the first atrial fibrillation episode and ‘zoom in’ for closer inspection. This illustrates the onset of the atrial fibrillation episode and shows that it begins with an instance of first-degree atrioventricular block (AV block), or PR prolongation, as shown by the increased delay between the P-wave and the R-wave in these traces, just before the onset of the atrial fibrillation. First degree AV block is clinically defined as the prolongation of the PR interval on the ECG to more than 200 msec—this can be seen and diagnosed in an ECG trace of the identified time period of interest.

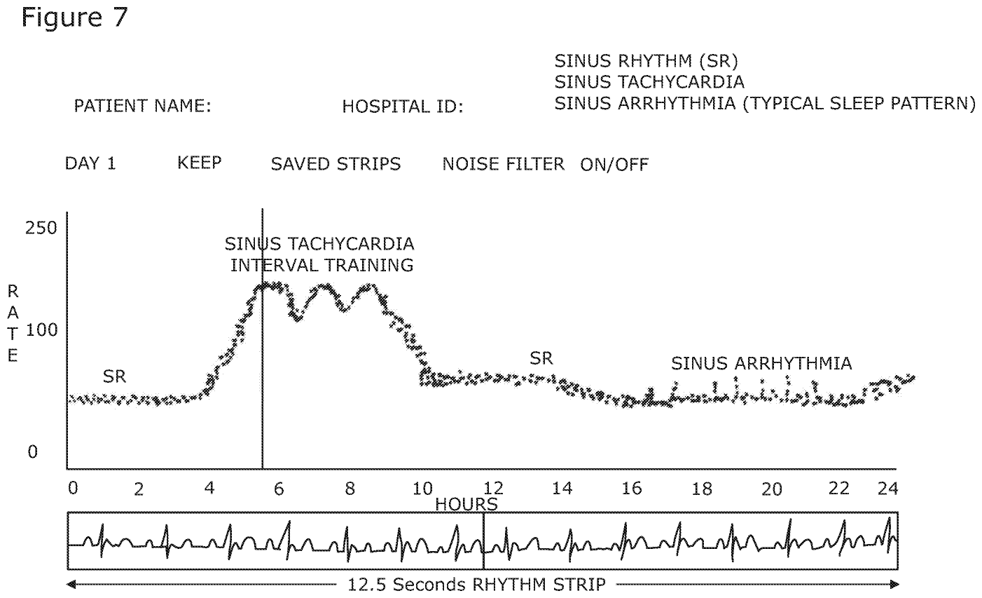

FIG. 7 shows an illustration of cardiac data over a 24 hour period with a mixed pattern of primarily sinus rhythm but with episodes of tachycardia. There is a prolonged period of tachycardia which is shown to be of gradual onset, with several peaks of heart rate visible, which could relate to ‘interval training’ exercise on behalf of the subject. In the later part of the trace, likely corresponding to sleep of the subject, episodes of sinus arrhythmia can be seen—with periodic transient increases of the heart rate. Sinus arrhythmia can be caused by the relationship between ventilation and heart rate, but can also indicate certain disorders.

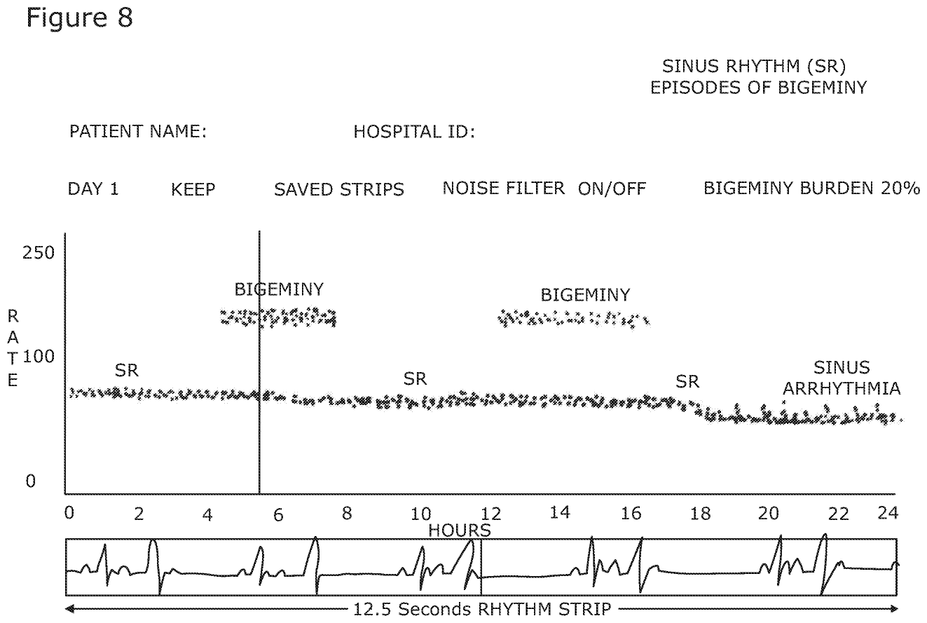

FIG. 8 shows an illustration of a 24 hour R-R plot with episodes of bigeminy and sinus rhythm—a bigeminy burden of 20% over this time frame. Additionally the trace shows instances of sinus arrhythmia during part of the trace. Bigeminy is clear from the R-R plot as two distinct lines present at the same time during the trace. As shown in the lower panel, bigeminy may be confirmed by viewing the ECG at one of these points, indicating ECG patterns coming in pairs, with longer intervals between consecutive pairs. Sinus arrhythmia can also be seen towards the end of this time period.

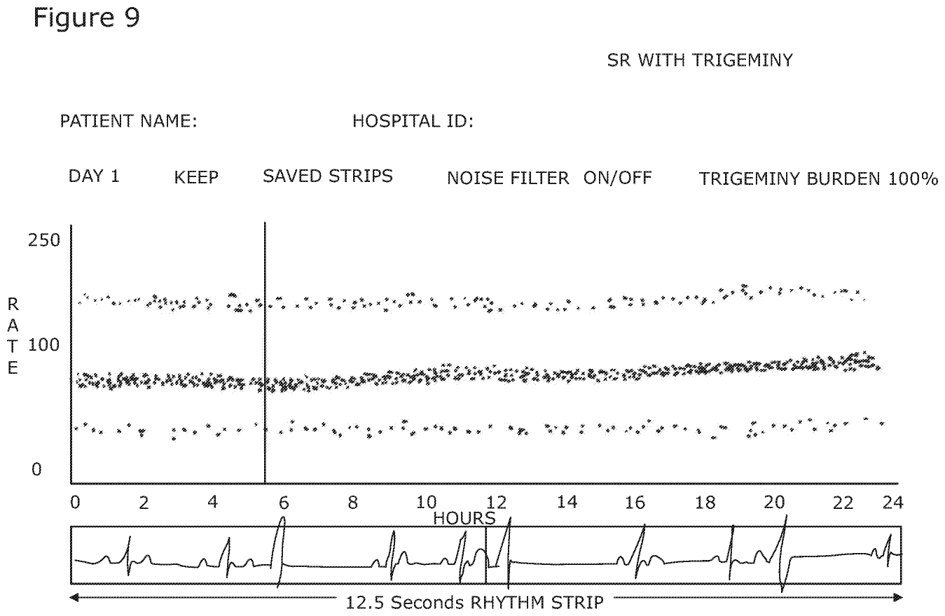

FIG. 9 shows an illustration of a 24 hour R-R plot with continual trigeminy—a trigeminy burden of 100%. This is shown by the three clear lines visible in the R-R plot. As shown in the lower panel, trigeminy may be confirmed by viewing the ECG at one of these points, indicating ECG patterns coming in distinctive triplets, with different intervals between each ECG trace, and longer intervals between each triplet.

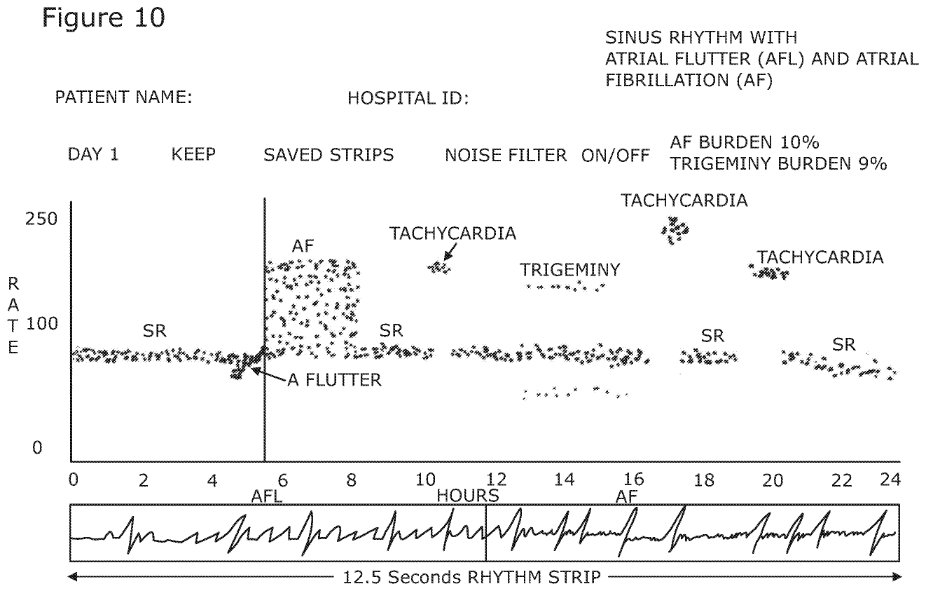

FIG. 10 shows an illustration of a 24 hour R-R plot with a base level of sinus rhythm and episodes of atrial flutter, atrial fibrillation, rapid onset tachycardia and trigeminy. The lower panel shows ECG data from the onset of the atrial fibrillation episode, and the end of the atrial flutter period.

### Data Handling and Upload

In some aspects the invention relies on a controller comprising input and output means, and processing means arranged at least to identify, within the first cardiac data, events corresponding to ventricular contraction (e.g. R-waves where the first cardiac data is ECG data, and pulses where the first cardiac data is pulse data), and the time at which they occur, and to produce second cardiac data in dependence on the intervals between said events. Different components of the controller of the invention may be located in one place or several, and may be local or remote from any users of the invention. For example, in some embodiments of the invention, the identification of events corresponding to ventricular contraction, the determination of intervals and production of second cardiac data may be carried out on a cloud-based platform, whereas the display means may be a device local to a user of the invention.

The controller may comprise one or more memory banks (e.g. one or more data stores) for storing information useful to the invention. For example, cardiac data may be stored if it relates to longer term recordings. Information relevant to the analysis of the second cardiac data may also be stored, for example standard profiles of normal heart rate data, thresholds of rates of change of heart rates, statistical features corresponding to particular heart rhythms, or templates of particular heart rhythms.

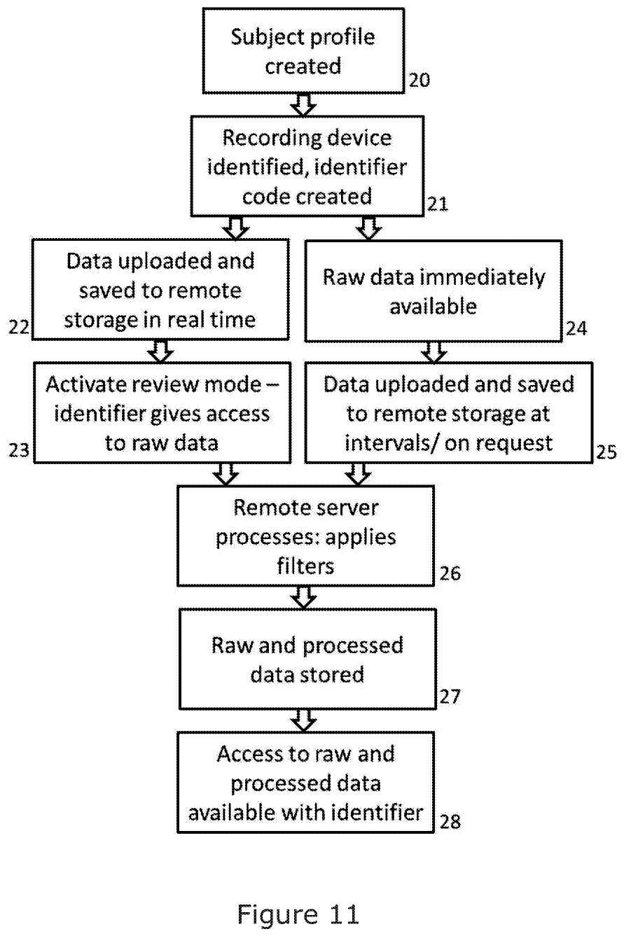

FIG. 11 shows a flow chart summarising the process of data storage and access of the raw and processed ECG cardiac data in embodiments of the invention. The data processing and storing steps, and the hardware to carry them out, may be local to the subject, for example comprised within the device which records the cardiac data, or may be distributed centrally or on a cloud-based platform, to which communication may take place through wired or wireless means, as appropriate. In particular, in this regard the process may involve a personal computer, mobile telephone or ‘smart’ device, and/or a remote server.

A profile for a person is created locally or centrally (20) with information which may include individual identifiers, such as a social security number or equivalent, hospital number, tax file number, and so on. The device which records the cardiac data is given its own identifying information and is matched with this profile (21). In this way, a unique pairing can be created such that there is no need to upload personal information, which may be sensitive, with cardiac data.

In arrangements where the cardiac data is analysed and stored remotely to the subject, two exemplary approaches are described below. In ‘real-time’ upload mode, raw data is uploaded continually (in ‘real time’) or at short intervals to the remote storage (22). When the subject requests access to the raw data, this is downloaded from the remote storage (23), using the match between the local device and the subject profile. In ‘online upload mode’, raw data is initially stored locally to the subject, such as on the recording device or an associated personal device such as a mobile telephone. Raw data may be available locally to the subject or other user for immediate use (24), before upload or permanently. Upload to the remote storage takes place at longer intervals and/or on request (25) of the subject or user.

After upload to the remote storage, data analysis takes place (26), which may be as discussed below, including the application of necessary filters in order to remove sources of noise and to identify the individual R waves from ECG data. Creation of R-R plots or other information also takes place. Both raw and processed data may be stored remotely, and/or returned to a local device for use by the subject or user, with access permitted by identifying information as set forth above (28).

### ECG Trace Analysis and Identification of Waveforms

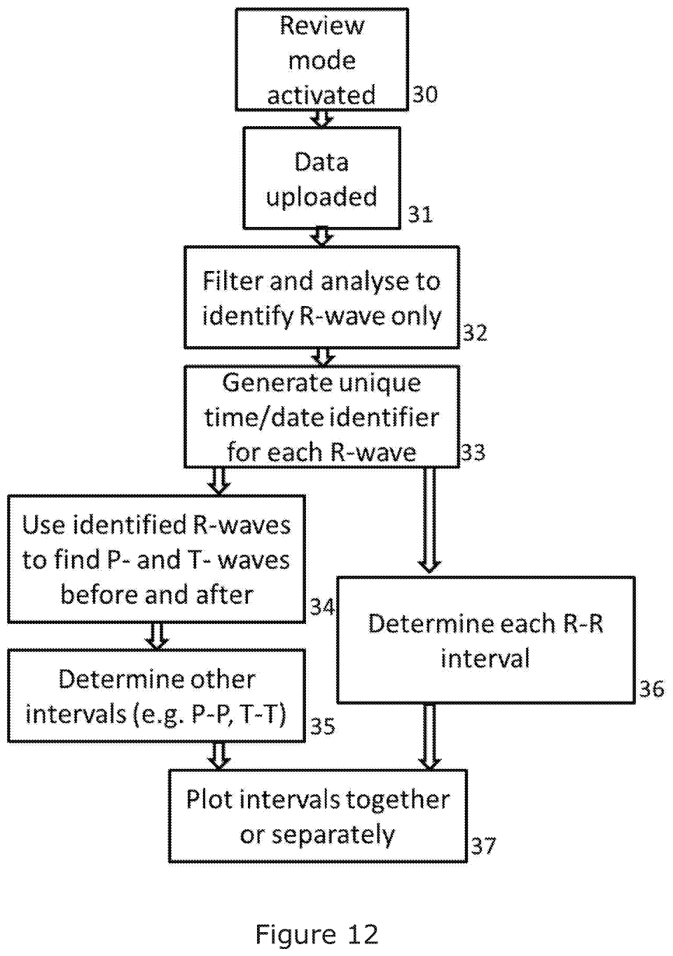

FIG. 12 shows a flow chart summarising the process of analysis of the first cardiac data comprising ECG data to produce the interval data for use in embodiments of the invention.

When review mode is activated (30), collected ECG data is uploaded (31), if appropriate and not already uploaded, to the data analysis controller. Filters and analysis are then carried out to identify the R-waves present in the ECG trace (32).

By using only the largest amplitude signal in the cardiac cycle (the R wave, corresponding to the ventricular beat) specific, tailored filters can be relatively easily applied in order to remove unwanted noise, providing a clean signal for analysis).

Filters contemplated for use are intended to include any suitable filters known in the art. Multiple scans can be carried out and/or multiple filtered traces can be generated at this stage in order to determine different aspects of the ECG trace. For example, a low pass filter will allow slow moving frequencies (such as the P wave) to be seen while the fast moving QRS complex to be hidden. A high pass filter will permit the reverse, while tailored band pass filters allow for a mixture between the two. A slew filter can look at the change in amplitude over time and only allow low amplitude, slow moving signals to be passed. Any negative frequencies can be removed. Filters which are particularly contemplated for use in removal of noise include notch filters to remove activity derived from any electrical background (for example mains electricity at 50 Hz or 60 Hz), and from muscle movement. In this way, different aspects of the recorded trace can be identified separately, and then recombined afterwards to show any interrelations, and to synchronise with the raw ECG data, as desired.

Filters will also be used to identify the R-wave/QRS complex from the ECG data. As the largest amplitude signal, one or more of many suitable techniques may be used for this identification, for example a trigger threshold set at a certain amplitude, a gradient analysis to find the fastest rates of change found during the ECG, or the use of a ‘template’ wavelet to identify waveform shapes which resemble a typical QRS complex. Several software approaches are known for the automatic identification of the components of ECG data (such as NEMon software, as discussed above).

It is envisioned that approaches which are known in the art may be used in order to apply filters to remove noise and isolate ECG features. Examples of this may be found for example in references 3 to 10 as listed below.

The advantages of using the R waves to identify an R-R interval, compared to using another part of the ECG trace such as the P-wave, is the relative ease of identifying these peaks over the rest of the trace and any noise which may be present. This means that this feature is particularly suited to automatic detection and analysis, allowing mass review of the data from one or more than one subject at once, with relatively limited human input.

In embodiments where monitoring devices that utilize multiple inputs of data (for example photo-sensitive technologies (PPT), pulse oximetry, oscilloscopes etc.) are used, these multiple inputs can also be used to aid in the discrimination of noise from real cardiac signals. Thus for example an apparent R-wave on the ECG trace which is not accompanied by a pulse as measured by a peripheral pulse oximeter might be classified as a false-positive.

Each R-wave is then given a unique identifier (33) marking its time, date and the subject from which it initiated. The R-wave once identified can also be used as a marker for the ‘mid-point’ of an ECG trace, and so by tracking forward and backward in time from the identified timepoint of an R-wave, the likely timepoints of P- and T-waves can be found, aiding in their identification.

P- and T-wave information may likewise be used in order to produce plots of P-P or T-T intervals. This has the potential of providing additional information, such as the refractory speed achieved by the cardiac tissue, that is, the time taken for a cardiac cell to depolarise, repolarise and be ready to fire again, as a summation of all cells in the same area of the heart. For the ventricle tissue, this is given by the R-T interval. In addition, mismatches between the results provided using each of these types of interval data can be informative. For example, in atrial fibrillation, P-waves are often absent—as a result a correlation between R-wave and P-wave data for the same time period would show a mismatch, suggesting possible atrial fibrillation.

As previously mentioned, it is also possible to use first cardiac data from other sources, that is, pulse data, to approximate the R-R plots or other ECG interval data used. Therefore it is contemplated that any of the methods as herein described may where possible also be applied to heart rate data gathered by means other than ECG. With this approach, however, it is not possible to retrieve ‘micro’ scale data (i.e. individual pulse ECG beat morphology), unless ECG data is concurrently recorded, and as such, ECG data and R-R plots are preferred.

In some embodiments, data from different sources can be combined for a single subject. For example, ECG data gathered from the subject in a diagnostic clinic or hospital setting can be combined with ECG data obtained over a different time period from a Holter monitoring device, and/or with pulse data recorded over another time period by a peripheral pulse detector. In this way, maximum data coverage for a particular subject can be achieved, even in times where, for example, ECG data cannot be conveniently recorded.

As mentioned, the combination of data from different sources can be used to improve the accuracy of the system. Such a possibility is reflected in FIG. 15. In this case ECG data (50) is collected, for example by a standard ECG 1-12 lead monitor. In addition, pulse wave velocity data (40) is measured with ultrasound sensors or similar, as discussed above, to provide supplementary cardiac data. These two sources of cardiac data can be correlated and compared, for example by plotting them on the same axis, as shown in FIG. 15. The peak of the pulse wave data (41) can be used to create a ‘blanking period’ (60) around the time of each trace (dashed box), denoting a time frame in which an ECG trace corresponding to ventricular contraction is expected. As discussed below, it may be necessary for this blanking period to be temporally offset from the peak which triggered it. If a peak (51) is detected in the ECG trace around this point, it is more likely that such an ECG peak corresponds to a genuine R-wave, and can therefore be accepted, and used to determine an interval, and thereby recorded as such on an R-R plot. Conversely, if no correlation is found between a putative R-wave and a blanking box triggered by a peak of pulse wave data, this can be used as evidence of a false positive, and the putative R-wave can be rejected. The temporal relationship between the pulse and ECG data will evidently vary based on a range of factors, most notably the body location of the sensor used to determine the pulse wave, but also for example on blood pressure and posture. For this reason, the nature, duration and temporal offset of the blanking period created by each peak may require a calibration step.

The correlation as described can also or alternatively be applied in reverse, such that a peak in an ECG trace can be used to generate a blanking period which is used to verify whether a putative pulse event from a pulse data source genuinely relates to ventricular contraction.

It can be appreciated that several types of cardiac data can be cross-correlated in a similar way to the above. Any combination of data sources can be used to reduce the risk of false positives. Additionally, in embodiments where the data which gives rise to the R-R interval data is not ECG data, but is pulse data collected for example by one or more of pulse oximetry, pulse photoplethysmography, blood pressure measurement and electrical measurement, it may be particularly advantageous to correlate several sources of data to improve accuracy.

As discussed above, pulse wave data, such as determined by ultrasound sensors, can in some circumstances be used to determine pulse wave velocity (PWV) information, and thereby blood pressure. In particular, an external central blood pressure system or a wearable external pulse wave/blood pressure system can be used, comprising several devices located in various places in the body that monitor a plurality of blood vessels, such as the carotid, radial, brachial and iliac arteries. Such a system can give multiple pulse wave forms from different body locations, such as the arteries mentioned above. These pulse wave forms can be correlated with each other and/or with an ECG trace, for example by overlaying the wave forms, creating a blanking period based on the detection of pulse onset, and template matching this to a putative R-wave, as described above, to provide evidence for classification as a genuine R-wave or otherwise.

The analysis will be able to be conducted using both stationary and mobile technology, allowing the analyst to be able to move quickly from a ‘macro’ overview to zoom in more and more to obtain increased ‘micro’ detail, which may include ECG data such that individual beat morphology can be seen to provide and capture highlighted segments of customizable duration for the digital and hard report.

The goal of the analysis software is to provide a multifunctional platform that accepts data from all types of cardiac and pulse monitoring technologies both medical and recreational, both in review and real time modes to provide macro and micro diagnostic information of an individual's cardiac performance over a customizable duration and represent the information in an easily identifiable and clinically meaningful manner, that is, in an R-R plot.

When used in combination with portable methods of heart rate and/or ECG monitoring, such as those utilised by the recreational user, including ‘wearable technology’, it is contemplated that real-time analysis of the first cardiac data may be used as each beat is recorded, and the interval between beats is calculated. By this method, it will be possible to provide notifications to the user or a third party, such as a healthcare professional, when certain features are detected in the R-R plot, such as changes in cardiac performance, cardiac rhythm, heart rate variability and/or changes in heart rate over time and during periods of increased heart rate variability.

This is particularly important for the screening, identification and/or monitoring of acute rhythmic disturbances such as episodes of atrial fibrillation. By being able to compare previous recordings and immediately calculate heart rate and heart rate variability, the user can be informed or ‘warned’ of potentially clinically significant changes in cardiac performance that may require medical management or increased medication dosing. Such warning may be, for example, by an audible, visible or tactile signal, via SMS, email or otherwise.

In this and any other embodiment, provision of the invention in a personal device such as a personal desktop or laptop computer or particularly on a mobile device such as a ‘smartphone’, tablet, or similar, may be advantageous. This may typically include a plurality of linked devices, which may communicate with each other by any suitable wired or wireless means, including infrared, wireless internet networking, mobile telephone network, radio and otherwise. For example, a device for measuring ECG data and another device for measuring pulse data may communicate said data to a mobile device for storage and analysis. Where the invention comprises a computer program, this may be in the form of an application (‘app’) which would permit convenient access to the invention on one or more personal and/or mobile devices.

In the same context, as a clinical research tool the software in real time mode can be used to monitor the effects of cardiac and non-cardiac pharmacological preparations or other treatments to monitor for changes in cardiac performance over time. In this way, each subject can act as their own control.

For the recreational user, the recordal of data of this kind can be used to monitor the response of the heart rate to exercise, and may be configured for example to inform the user the proportion of time that their heart rate was elevated over a particular value, during a certain time period, or to monitor resting heart rate. This is of use for those who are interested in monitoring particular fitness goals, such as the reduction of a resting heart rate or the maintenance of an elevated heart rate for a certain length of time per week.

It is considered that further analysis of the second cardiac data may in some embodiments be of use in identifying areas of the plot which may be of interest, and thereby ‘highlighting’ them for later attention by the user of the data, or for the provision of ‘warning’ signals as discussed previously. This may be done by categorising the second cardiac data into one or more rhythmic categories corresponding to heart rhythm or arrhythmia types. For example, the heart rate as measured by the R-R plots could be grouped by frequency over a particular period, and the statistical features of these data used to indicate particular rhythm disturbances. The statistical features associated with particular rhythmic types could be stored by the invention, and used for comparison with the second cardiac data. For instance, atrial fibrillation would be characterised by a large variance and no clear mode, and bigeminy and trigeminy would be respectively bimodal and trimodal.

Calculating the rate of change of a heart rate over time may also be used in detecting sudden or rapid onset conditions, and therefore may also be used to identify regions of interest. For example, one or more thresholds may be set for a rate of change of heart rate, above or below which the plot would be flagged as an area of interest and/or as a boundary between two putative rhythmic categories. As an example, a pathological tachycardia event (for example caused by atrial fibrillation or atrial flutter) is characteristically sudden onset. This contrasts with tachycardia brought on naturally by exercise which will be of more gradual onset. Therefore, instances with a rapid change in heart rate may be selected by the analysis program for highlighting for later review.

The second cardiac data may also be compared to a standard profile of a ‘normal’ heart rhythm, which may be in particular a sinus rhythm, which may be taken from the same subject from which the first cardiac data is taken, or may be from another subject, or an artificially generated profile. Thus a deviation from such a standard profile may indicate a deviation from a ‘normal’ heart rhythm, and therefore the presence of a rhythmic disturbance.

Similarly, the second cardiac data may be categorised into one or more rhythmic categories by comparing to stored templates corresponding to said categories. For example, a stored template could be second cardiac data corresponding to an episode of atrial fibrillation, which again may be from the same or a different subject, or could be artificially generated. Similarity of any portion of the second cardiac data to this template would indicate that the portion should be categorised accordingly. Multiple templates could be stored, corresponding to different rhythmic categories.

The identification of periods of interest or of categorisation into different rhythmic categories could involve the generation of additional data and/or particular data processing steps, which may include average rate, rate of change, standard deviation, mean squared differences, Fourier transforms, clustering algorithms, and other techniques. An approach of using of movable or ‘rolling’ time ‘windows’ of varying sizes may be used to detect periods of distinct activity for the above analyses. For example, the interval data may be analysed in multiple overlapping windows of 1 hour, which would allow for the identification of rhythmic disturbances lasting at least an hour.

This approach can be used particularly effectively when paired with individual subject data, so that if a subject is known to be affected by a particular condition, this further analysis can look for indications of episodes of that condition in particular, and highlight them for attention of a user such as a medical professional. Information such as expected number of episodes per day, average duration of episodes, and relative severity of the condition can be used to further refine this analysis process and so improve the information provided to the subject and/or the user. When combined with a learning algorithm, this approach can be improved over time, as more information about a subject's particular cardiac function is gathered. When analysis occurs in real-time or at frequent intervals, identification of episodes can be used by the system of the invention to provide an output signal which may be an alert which is presented to a subject and/or user of the system by any suitable means, for example by email, SMS or an app update on a ‘smart’ device. It is contemplated that a period of calibration may be used, for example of two weeks' duration, at the beginning of a subject's use of the device according to an embodiment of the invention.

Such correlation with a subject's individual profile can also be used to track the evolution of a particular condition over time. As an example, on primary diagnosis a patient may exhibit an atrial fibrillation burden of 1%, that is, they are experiencing an atrial fibrillation episode 1% of the time. The patient can be issued with a device such as a Holter device or heart rate to monitor the primary cardiac data. The measuring device is able to transmit information in real time or at short intervals to a processing means, and processing and analysis of the data can therefore begin at once, which can take the form of applying any filters to isolate markers of ventricular contraction (e.g. R waves in the ECG or pulses) from the raw first cardiac data, and producing preliminary display plots for later viewing. These plots and analysed data are compared to previous recordings, in order to detect any change. Over time, such as months or years, the atrial fibrillation burden may increase to 2% or higher, which can be detected by these retroactive comparisons. Other changes can be detected, such as an increase in the heart rate exhibited during the episodes. Recordal of data during this time will thus be able to track this increase and alert the patient or a medical practitioner to any increases without repeated diagnostic visits, and may inform treatment changes, such as the necessity of providing medicine to lower heart rate, as well as anticoagulants. Similarly, changes in the length and/or frequency of episodes may be detected in this way. This approach may be particularly useful when used to monitor the effect of particular treatments, such as a change in a pharmaceutical strategy with a particular patient—it can be seen how such an intervention affects the patient over a longer period of time.

It is envisaged therefore that a learning process may occur, wherein the analysis of the second cardiac data is updated over time to better fit a particular subject, or a particular heart rhythm type or category. The nature of this learning process will depend on the particular way in which the second cardiac data is analysed, for which examples are given above. Thus, learning may occur by updating a threshold for the rate of change of heart rate, by updating the statistical features associated with a particular rhythmic category, or by updating to standard profile or the stored templates to which the second cardiac data is compared.

In addition, the learning process may make use of human input. A medical practitioner may analyse the beat-to-beat plot, with or without the automatic analysis, and categorise the data according to one or more rhythmic categories. The automatic analysis would then be updated through the learning process by one of the above methods, or otherwise, in order to more closely resemble the analysis of the medical practitioner. This would allow the detection to be changed to be more or less sensitive and/or selective, thereby reducing false negatives or false positives.

It is contemplated that the cardiac data used by the invention can be coordinated with other data collected in real time from a subject. For example, for subjects who have a pacemaker fitted, data from the pacemaker can be gathered at the same time as the cardiac data to ensure that cardiac activity is matching the output of the pacemaker. Failure to capture, that is, when the heart tissue fails to respond to a pacemaker impulse, could be identified by this approach, and a subject and/or user informed.

Monitoring of the subject's ventilation activity (breathing in and out) can also be used in concert with the cardiac data in order to measure and/or control for well-known effects of inspiration and expiration on heart rate (that is, heart rate is known to generally increase slightly during inhalation, and decrease slightly during exhalation).

Other data which may be correlated with the cardiac data used by the invention may include galvanic skin response, body temperature, posture, blood pressure and vascular tone. Changes in these data may correlate with particular heart rhythms (or vice versa), thereby allowing improved accuracy in the detection of any episodes of rhythmic disturbance.

For example, blood pressure measurements can be derived from pulse wave monitoring as discussed above, or otherwise. This information can then be provided to a user of the system in conjunction with the R-R plots and/or ECG data gathered at the same time. In this way, when a user zooms in on a particular time point, all available data can be reviewed, potentially including: ECG waves (such as recorded by 1-12 Lead ECG); pulse waves (from single or multiple locations); central blood pressure, and any other data such as Body Temperature and body position (supine, standing etc). The above would allow patients, physicians and/or other users to review the health of the subject in more detail, and from a single beat to more long term analysis. The pulse wave interval data linked to central blood pressure information allows for evaluation to determine and take into account into hyper- and hypotension. This information can provide further evidence for certain conditions.

For instance, postural orthostatic tachycardia syndrome (PoTS) is a condition where a large increase in heart rate results from a change in posture from lying or sitting to standing, but is not necessarily associated with a drop in blood pressure. In this regard it can be contrasted with orthostatic hypotension, which is a drop in blood pressure on similar postural changes, although both conditions can result in syncope (fainting) and other symptoms. Episodes of such conditions, as well as the difference between them, can be seen with reference to a system in accordance with the invention, in particular where blood pressure information is also available. PoTS can be indicated by a rapid increase in heart rate, and the presence or absence of hypotension can be noted. If available, other recorded subject data from the same time points such as posture or galvanic skin response can be used to indicate these conditions. In this way, episodes of the above-mentioned conditions can be distinguished from other cardiac events.

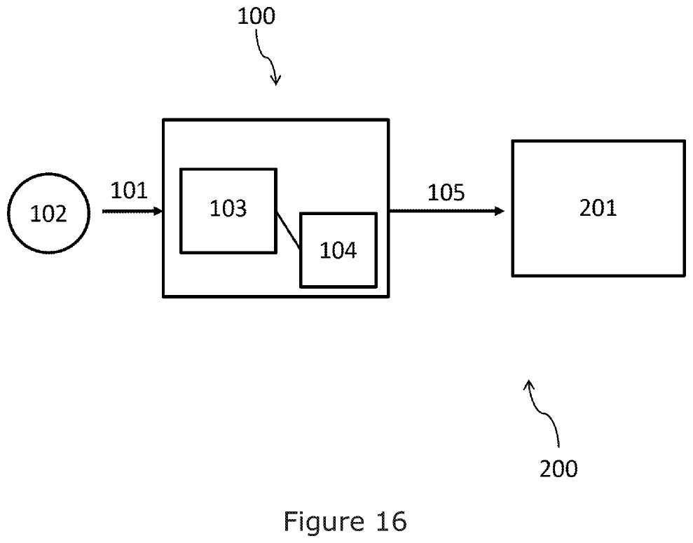

A schematic of a controller and a system according to an embodiment of the present invention is shown in FIG. 16. This Figure shows a controller (100) comprising input means (101) for cardiac data (102); processing means (103), and output means (105). The controller (100) further comprises a memory (104). The controller (100) is configured to carry out the methods and/or processes as described for example in relation to FIG. 12. A system (200) according to an embodiment of the present invention further comprises a display means (201) in addition to the controller (100).

The aforementioned embodiments are not intended to be limiting with respect to the scope of any claims, which may be filed on applications filed in the future and claiming convention priority from this application. It is contemplated by the inventors that various substitutions, alterations, and modifications may be made to the invention without departing from the spirit and scope of the invention as defined by the claims.

### REFERENCES

1. Tan A Y, Ellenbogen K A, Kaszala K, Huizar J (2015) Sustained Multiple Railroad Tracks on Implantable Cardiac Defibrillator Interval Plots, Mechanisms and Management; Circulation: Arrhythmia and Electrophysiology. 8:1284-1288 https://doi.org/10.1161/CIRCEP.114.002633

2. AlGhatrif M, and Lindsay J. (2012) A brief review: history to understand fundamentals of electrocardiography, Journal of Community Hospital Internal Medicine Perspectives, 2:1, DOI: 10.3402/jchimp.v2i1.14383

3. Sharmila V and Reddy K. (2015). Identification of Premature Ventricular Cycles of Electrocardiogram Using Discrete Cosine Transform-Teager Energy Operator Model. Journal of Medical Engineering. 2015. 1-9. 10.1155/2015/438569

4. Brown L F, Arunachalam S P (2009) Real-time T-p knot algorithm for baseline wander noise removal from the electrocardiogram, Biomed Sci Instrum.; 45:65-70.

5. Akwei-Sekyere (2015), Powerline noise elimination in biomedical signals via blind source separation and wavelet analysis, PeerJ 3:e1086; DOI 10.7717/peerj.1086

6. S. A. Taouli & F. Bereksi-Reguig (2009) Noise and baseline wandering suppression of ECG signals by morphological filter, Journal of Medical Engineering & Technology, 34:2, 87-96, DOI: 10.3109/03091900903336886

7. Taouli S A, Bereksi-Reguig F (2010) Noise and baseline wandering suppression of ECG signals by morphological filter J Med Eng Technol. February; 34(2):87-96. doi: 10.3109/03091900903336886.

8. Agrawal S, Gupta A (2013) Fractal and EMD based removal of baseline wander and powerline interference from ECG signals, Comput Biol Med. 2013 November; 43(11):1889-99. doi: 10.1016/j.compbiomed.2013.07.030. Epub 2013 Aug. 26.

9. Niegowski M, Zivanovic M, Gomez M, Lecumberri P (2015) Unsupervised learning technique for surface electromyogram denoising from power line interference and baseline wander, Conf Proc IEEE Eng Med Biol Soc.; 7274-7. doi: 10.1109/EMBC.2015.7320071

10. Bahaz M, Benzid R (2018) Efficient algorithm for baseline wander and powerline noise removal from ECG signals based on discrete Fourier series, Australas Phys Eng Sci Med. March; 41(1):143-160. doi: 10.1007/s13246-018-0623-1. Epub 2018 Feb. 5

1. A controller for monitoring cardiac data for the presence of arrhythmia, comprising:
input means arranged to receive first cardiac data corresponding to a time period;
processing means arranged to:
identify, within the first cardiac data, a plurality of events corresponding to ventricular contraction, and a time associated with each event;
determine a plurality of intervals between the times associated with chronologically successive events; and
produce second cardiac data in dependence on the determined intervals;
and output means arranged to transmit an output signal to a display means based on the second cardiac data, for displaying a beat-to-beat display plot corresponding to at least a part of the time period on the display means.

2. (canceled)

3. The controller according to claim 1, wherein the input means is arranged to receive a request to alter the part of the time period to which the beat-to-beat display plot corresponds.

4. (canceled)

5. The controller according to claim 1, wherein the first cardiac data comprises ECG data and wherein the processing means is arranged to identify a plurality of R-waves in the ECG data as events associated with ventricular contraction.

6. The controller according to claim 1, wherein the first cardiac data comprises ECG data and wherein the input means is arranged to receive a request to display the ECG data associated with a particular part of the time period.

7. (canceled)

8. The controller according to claim 1, wherein the input means is arranged to receive supplementary cardiac data corresponding to the same time period as the first cardiac data, the first and supplementary cardiac data corresponding to different sources of cardiac data; and
the processing means is arranged to
identify, within the supplementary cardiac data, a plurality of supplementary events corresponding to ventricular contraction, and a time associated with each supplementary event;
compare the identified events with the supplementary events to determine if a given event correlates with a corresponding supplementary event, and where a correlation exists for a given event, to use the identified event to determine the plurality of intervals between the times associated with chronologically successive events.

9. The controller according to claim 8, wherein the first cardiac data and supplementary cardiac data correspond to ECG data and pulse data respectively.

10. The controller according to claim 8, wherein the processing means is configured to reject events corresponding to ventricular contraction where a correlation does not exist for a given identified event and a corresponding supplementary event.

11. The controller according to claim 1 wherein the processing means is arranged to analyse at least the second cardiac data to detect the presence of one or more episodes of arrhythmia.

12. The controller according to claim 11, wherein the processing means is arranged to undergo a learning process to refine the analysis of at least the second cardiac data.

13. (canceled)

14. The controller according to claim 11, wherein the processing means is arranged to identify a deviation of at least the second cardiac data from a standard profile.

15. controller according to claim 14, wherein the processing means is arranged to carry out a learning process comprising updating the standard profile.

16. The controller according to claim 11, wherein the processing means is arranged to identify a rate of change of the calculated heart rates over time above a threshold.

17. The controller according to claim 16, wherein the processing means is arranged to carry out a learning process comprising updating the threshold.

18. The controller according to claim 11, wherein the processing means is arranged to categorise at least the second cardiac data into one or more rhythmic categories according to stored templates or statistical features corresponding to said rhythmic categories.

19. The controller according to claim 18, wherein the processing means is arranged to carry out a learning process comprising updating the stored templates or statistical features.

20. (canceled)

21. A method of analysing and monitoring cardiac data for the presence of arrhythmia, the method comprising:
obtaining first cardiac data;
identifying within the first cardiac data a plurality of events corresponding to ventricular contraction and a time associated with each event;
determining a plurality of intervals between the times associated with chronologically successive events; and
producing second cardiac data in dependence on the determined intervals;
outputting, based on the second cardiac data, an output signal based on the second cardiac data to a display means for displaying a beat-to-beat display plot corresponding to at least a part of the time period on the display means.

22. (canceled)

23. The method according to claim 21, further comprising a step of displaying, based on the output signal, a beat-to-beat display plot corresponding to at least a part of the time period and a step of displaying further data selected from galvanic skin response, body temperature, posture, blood pressure and vascular tone data, the further data corresponding to the time period of the beat-to-beat display plot.

24. The method according to claim 22, further comprising altering the part of the time period to which the beat-to-beat display plot corresponds, based on a received request.

25-27. (canceled)

28. A system for monitoring cardiac data for the presence of arrhythmia, comprising a controller for monitoring cardiac data for the presence of arrhythmia, and a display means for displaying the beat-to-beat display plot corresponding to at least a part of the time period, wherein the controller comprises:
input means arranged to receive first cardiac data corresponding to a time period;
processing means arranged to:
identify, within the first cardiac data, a plurality of events corresponding to ventricular contraction, and a time associated with each event
determine a plurality of intervals between the times associated with chronologically successive events; and
produce second cardiac data in dependence on the determined intervals; and
output means arranged to transmit an output signal to a display means based on the second cardiac data, for displaying a beat-to-beat display plot corresponding to at least a part of the time period on the display means.

29-30. (canceled)

31. The system according to claim 28, further comprising at least one device for collecting first cardiac data.

32. The system according to claim 31, wherein the first cardiac data comprises ECG data, and the device is selected from: a Holter monitor, a cardiac patch, a 12-lead ECG, a 5-lead ECG, a 3-lead ECG; a loop recorder and an implantable loop recorder.

33. (canceled)

34. The system according to claim 31, wherein the first cardiac data comprises pulse data and the device collects said pulse data by one or more of: pulse oximetry, pulse photoplethysmography, blood pressure measurement and electrical measurement.

35-37. (canceled)
